(19) United States
(12) Patent Application Publication
(10) Pub. No.: US 2022/0118218 A1
(43) Pub. Date: Apr. 21, 2022

(54) SYSTEMS AND METHODS FOR REMOTELY CONTROLLED THERAPY

(71) Applicant: BioSerenity, Paris (FR)

(72) Inventors: William Louis Frederic Bense, Bagneux (FR); Pierre-Yves Frouin, Atlanta, GA (US); Raymund Schwan, Villers-Les-Nancy (FR); Thomas Bruno Schwitzer, Nancy (FR)

(73) Assignee: BioSerenity, Paris (FR)

(21) Appl. No.: 17/071,304

(22) Filed: Oct. 15, 2020

(51) Int. Cl. A61M 21/02; A61H 5/00; G06F 3/01; A61M 21/00; G06F 3/14; A61B 3/113; G06F 3/16

(52) U.S. Cl. CPC A61M 21/02; A61M 2230/10; G06F 3/013; A61M 2230/42; A61M 2230/435; A61H 2230/105; A61M 2021/0044; A61H 2201/1604; A61H 2201/165; A61H 2230/065; A61M 2209/088; A61M 2230/04; A61M 2230/432; A61H 2230/405; A61H 2230/425; A61H 2230/505; G06F 3/14; A61B 3/113; A61H 2201/501; A61H 2230/045; A61M 2021/0027; A61M 2230/50; G06F 3/16; A61M 2205/3584; A61H 5/00

(57) ABSTRACT

Systems and methods for providing remotely controlled therapy are provided herein. A therapy device connects to a therapy server over a network. The therapy device can include a headset and an audio device. A therapist can select one or more therapies to apply to a patient, including multiple therapies at the same time. The therapist can receive feedback from the patient that allows the therapist to adjust the therapies being applied.

### BACKGROUND

When seeking treatment for various afflictions, such as post-traumatic stress disorder (PTSD), conventional treatment methods often include the use of a therapist to perform various treatment therapies. Some of these therapies include audio, visual, light, smell, and the like.

It is with these and other concerns that an improved system and method for therapies is described herein.

### BRIEF DESCRIPTION OF THE DRAWINGS

The detailed description is set forth with reference to the accompanying figures. The use of the same reference numbers in different figures indicates similar or identical items or features.

FIG. 1 is an illustration of an example treatment system, in accordance with some examples of the present disclosure.

FIG. 2 is an example therapy headset, in accordance with some examples of the present disclosure.

FIG. 3 illustrates the use of a cellular telephone for use as a display for the therapy headset, in accordance with some examples of the present disclosure.

FIG. 4 is an illustration of a therapy computing device for use with the systems and methods described herein, in accordance with some examples of the present disclosure.

FIG. 5 is a flowchart depicting a process of using a therapy device, in accordance with some examples of the present disclosure.

FIG. 6 illustrates some example patterns that may be displayed on a display of a therapy headset for a therapy, in accordance with some examples of the present disclosure.

FIG. 7 illustrates the movement of a cursor to provide patterns that may be displayed on a display of a therapy headset for a therapy, in accordance with some examples of the present disclosure.

FIG. 8 is an illustration of an example treatment system using a condition detector in conjunction with a therapy device, in accordance with various embodiments of the presently disclosed subject matter.

### DETAILED DESCRIPTION

Examples of the present disclosure can comprise systems and methods for remotely controlled therapy. As used herein, “remotely controlled” means that a therapist or other person controls a therapy device installed on a patient. In one example, a headset is provided that allows a therapist to control various environment aspects of the user (client or patient—used interchangeable herein) wearing the headset. The headset may include various visual, light, and audio outputs that can be used by a therapist to administer a therapy. In some example, the therapist may be able to control visual cues, and in some instances, provide audial instructions or input for a therapy session. In one example, the headset may be a fully integrated unit that includes all functionality needed to establish communications with a device a therapist uses to administer a therapy. In another example, the headset may be configured to use additional devices. For example, the headset may be in communication with a user equipment (such as a cell phone) or the headset may be configured to hold the user equipment in visual proximity to the eyes of the patient undergoing therapy. In additional examples, the headset and/or a user device (such as a cell phone) may be configured to execute an application to administer a therapy.

The therapist, at the therapist workstation or other device, send instructions to have the headset display various types of visual images. The therapist may also be in audible communication with the patient, asking the patient to describe various events, thoughts, and the like while the headset is displaying the various types of visual images. In some examples, the therapist may be able to control other aspects of the headset to provide different types of therapy input. Some examples include, but are not limited to, luminotherapy, sound therapy, and the like. In addition, other forms of therapy may be used, including relaxation therapy whereby sound and/or an image are used to attempt to invoke a relaxing experience in the patient. Some additional examples include heart (cardiac) coherence and neurofeedback therapies. In some examples, cardiac coherence therapy uses biofeedback to control heart rate variability (HRV). The patient can use the biofeedback for relaxation and may be used as a stress management tool. In some examples, neurofeedback (or electroencephalogram) biofeedback, is a therapeutic technique that uses feedback to assess a patient's brainwave activity. The device then uses sound or other visual signals in an attempt to modify, or reorganize, these brain signals. The patient can learn how to regulate and improve these brain signals to reduce the effects of various disorders.

The use of a remotely controlled device according to various examples of the presently disclosed subject matter may provide various advantages over current systems. For example, patients suffering from various afflictions, including PTSD, may find it difficult to go to the therapist's location because of their affliction. Further, those needing therapy may not have available to them the means or ability to travel, such as those in remote locations or without a reliable means of transport. Additionally, in certain conditions in which the patient and/or the therapist are isolated (such as during a pandemic or viral outbreak), it may be undesirable for the therapist and the patient to be in the same room. Thus, the various examples of the presently disclosed subject matter may overcome limitations and disadvantages of conventional therapy.

An example of therapy that may be provided by the examples provided herein is Eye Movement Desensitization and Reprocessing (EMDR). EMDR therapy is an eight-phase treatment. Eye movements (or other bilateral stimulation) are used during one part of the session. After the therapist has determined which memory to target first, the therapist asks the patient to hold different aspects of that event or thought in mind and to use the patient's eyes to track the therapist's hand as it moves back and forth across the patient's field of vision. Another type of therapy is Eye Movement Integration (EMI). EMI uses guided eye movements to assist a patient to access recorded information in all its multisensory, cognitive and emotional forms. EMI recruits these resources to the integration and resolution of distressing experiences and their problematic psychological consequences. Another type of therapy that may be used with examples of the presently disclosed subject matter is light therapy (or luminotherapy). Light therapy is also known as bright light therapy or phototherapy. Luminotherapy uses various light intensities, colors, and the like for treatments. Uses of luminotherapy, heart (cardiac) coherence, and neurofeedback therapies include treatment for seasonal depression, jetlag, and cutaneous diseases.

Further, because the presently disclosed subject matter may use two or more types of therapies in a combined treatment, the effectiveness of the therapy device may be increased. The therapy may be adapted during the session based on answers provided by the user. Thus, instead of a passive device, various examples of the presently disclosed subject matter create an active and changing engagement with the user to enhance the effectiveness of the therapy in some examples.

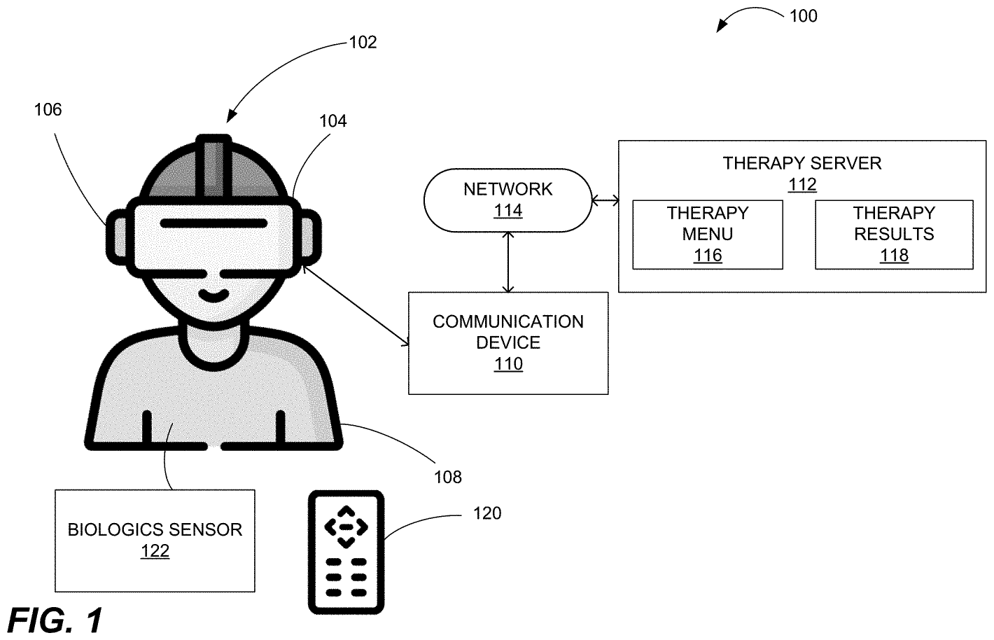

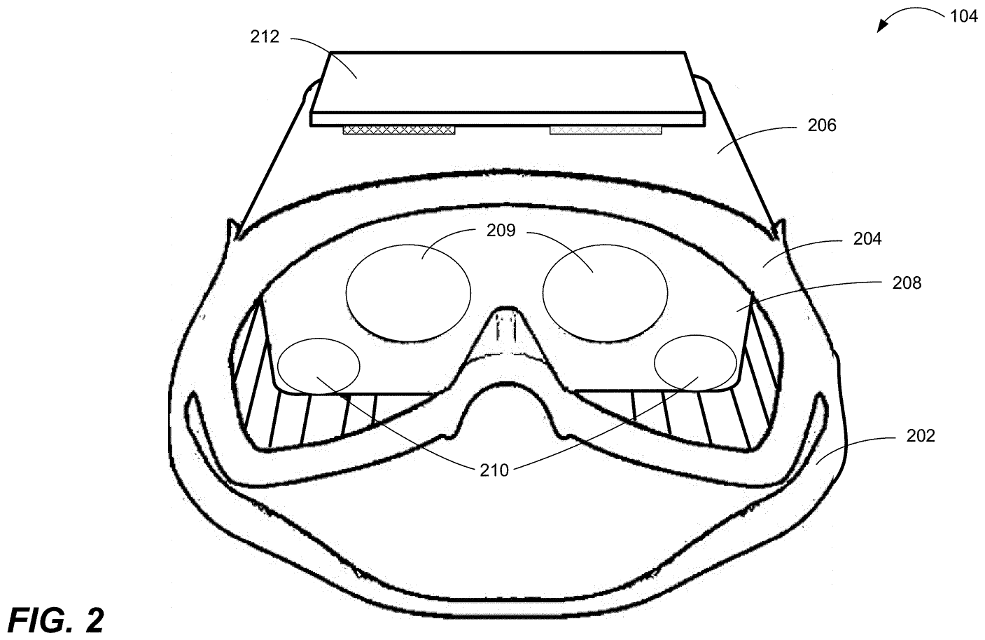

FIG. 1 is an illustration of an example treatment system 100, in accordance with various embodiments of the presently disclosed subject matter. Illustrated in FIG. 1 is a therapy device 102, explained in more detail by way of example in FIG. 2. The therapy device 102 includes a therapy headset 104 and a therapy audio device 106. The therapy headset 104 is designed to fit over a user's 108 head and partially or fully isolate the eyes of the user 108 from outside stimuli. The therapy audio device 106 is designed to fit over at least one if not both of the ears of the user 108 and partially or fully isolate the ears of the user 108 from outside stimuli.

The therapy device 102 is in communication with a communication device 110. The communication device may include an individual's cellular phone, tablet, a personal computer, and the like. The communication device 110 may be an integral part of the therapy device 102 or may be a separate piece of equipment that facilitates communication between the therapy device 102 and a therapy server 112. In some examples, a cellular phone may be integrated into, or added as a component to, the therapy headset 104 and may be used for various purposes, including as the communication device 110 or a screen. In some examples, the therapy device 102 is connected to a personal computer or tablet serving as the communication device 110. The communication device 110 may be connected using a wireless or wired connection to the therapy device 102. These and other examples are considered to be within the scope of the presently disclosed subject matter.

The communication device 110 receives and transmits information from and to the therapy server 112 through a network 114. The network 114 may be various types of networks that provide communication access between the communication device 110 and the therapy server. It should be noted that presently disclosed subject matter is not limited to the use of a particular type of network, including cellular networks, and communication device. The systems and methods discussed herein are discussed generally with respect to communication devices such as cellular UEs, tablets, computers, and the like, and in terms of components (e.g., network entities) associated with Wi-Fi networks, Bluetooth networks, wired networks, fourth-generation (4G) and fifth-generation (5G) cellular networks, and other types of networks. The systems and methods can be used with other types of equipment and on other types of networks, however, where users may wish to have increased flexibility in sending and receiving calls, video calls, and messages. Thus, the systems and methods described herein may be described in terms of the 4G and 5G networks merely because these networks represent the state of the current art. One of skill in the art will recognize, however, the systems and methods could also be used on other networks that provide video calling such as, for example, Internet of Things (IoT), machine-to-machine (M2M), sixth-generation (6G), and other current and future networks.

During use, a therapist or other personnel/computer software (not shown) may select one or more treatment options from a therapy menu 116. The therapy menu 116 can be established for the user 108 based on a predetermined treatment regimen or may include multiple therapies available. The therapy menu 116 may be displayed on a user device controlled by the therapist, whereby the therapist can select or deselect one or more therapies. Further, once the therapy device 102 is in communication with the therapy server 112, the therapy menu 116 may be updated based on the capabilities of the therapy device 102. For example, the therapy device 102 may only be capable of specific light, visual, or audible outputs, limiting the types of therapy that may be available. By updating the therapy menu 116 once connected, the therapist (or another person or computer program acting at the direction of the therapist) may have for selection from the therapy menu 116 those treatments that are available. Further, the treatment menu 116 may be updated based on specifics applied to a particular therapy. For example, it is usually not recommended to perform luminotherapy in the evening. Additionally, one or more therapies may be selected at any particular time for a combined therapy session. For example, EMDR or EMI may be selected along with luminotherapy as well as an audio therapy and/or a relaxation therapy, or various combinations thereof.

The therapies and their results may be stored as therapy results 118. The therapy results 118 may be an online or locally-stored record of data taken during past therapy treatments. The therapy results 118 may include, but are not limited to, the types of therapies, the patterns used in an EMDR or EMI session (or a similar eye movement session), when the therapies were performed, the location of the user 108 during the therapy, results of the eye tracking system, a percentage of patterns completed, any notes taken during a particular therapy session, and the like.

In an example of a therapy session, such as EMI, the user 108 may be asked a series of questions through the therapy headset 104. For example, the user 108 may be shown various patterns on a screen (not shown) of the therapy headset 104. The user 108 may be asked if the user 108 is better/worse/same after each pattern. The user 108 may answer using a remote control 120 which is in communication with the therapy device 102. In some examples, the user 108 may answer using head movements or eye movements. The therapy device 102 records and transmits the answers to the therapy server 112 for storage as the therapy results 118. In some examples, if the user 108 answer that they are worse after a particular pattern, the therapy server 112 may update the server menu 116 to prevent the particular pattern from being shown again. During a typical EMI or EMDR session, eye movement of the user 108 is controlled using a pointer on a screen and a message may be displayed on the screen of the therapy headset 104 if the therapy device 102 detects that the user 108 has stopped looking at the pointer.

In some examples, a biologics sensor 122 may be used to measure the user's 108 biological conditions. For example, the biologics sensor 122 may perform various analytical functions, including, but not limited to, electrocardiogram (ECG), Fc, heart rate variability (HRV), respiratory frequency, O2/CO2 expired, arterial pressure, electroretinography, electroencephalogram (EEG), neurofeedback, heart coherence, blood pressure, pulse rate, body temperature, and the like.

The biologics sensor 122 may be in communication with the communication device 110 to provide the measurements to the therapy server 112. Thus, answers or input provided by the user 108 may be analyzed against the measurements. This may be especially helpful to determine the truthfulness of answers or identify potentially stressful issues not realized or provided by the patient. The presently disclosed subject matter is not limited to one biologics sensor 122, as multiple biologics sensors 122 may be used.

FIG. 2 is an example therapy headset 104 that may be used in conjunction with various examples of the presently disclosed subject matter. It should be noted that although the headset 104 is illustrated as being a separate component from the therapy audio device 106, in some examples, the therapy audio device 106 and the therapy headset 104 are integrated into a single device. It should also be noted that one or more components of the therapy headset 104 may not be required and are merely provided as an example.

The therapy headset 104 illustrated in FIG. 2 includes a strap 202 for securement to the head of the user 108. The strap 202 may be of various flexible materials suitable for the purpose of securing the therapy headset 104 to a user's head. Additional straps or other strap designs, such as a strap across the top of the head of the user, may also be used and are considered to be within the scope of the presently disclosed subject matter. The therapy headset 104 may also include a cushion 204 fixed to a display enclosure 206. The display enclosure houses various components of a display and may be constructed of a material such as, but not limited to, plastic, foam, cardboard, and the like. The cushion 204 may be used to provide a comfortable interface between the user's head and the therapy headset 104. In some examples, the cushion 204, or other component of the therapy headset 104, may have integrated within the component sensors used to provide various biological measurements, such as those taken by the biologics sensor 122. The therapy headset 104 further includes partition 208. The partition 208 is used to provide an installation location for apertures 209 and eye movement trackers 210.

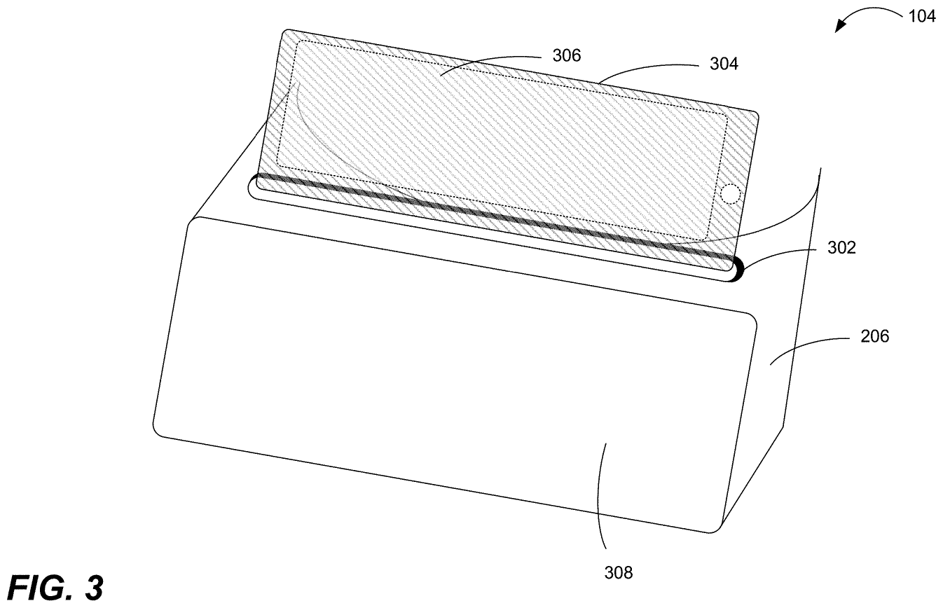

The apertures 209 are an opening through the partition 208 through which the user 108 may view a display (shown in FIG. 3). The apertures 209 may be holes or openings, or, may including one or more lenses or shutters. For example, the apertures 209 may be constructed similar to camera lenses whereby shutters in one or more of the apertures 209 may be opened or closed to provide additional therapy inputs. This may allow a therapist to remove visual inputs to the user 108 individually or in a specific pattern in one or both of the eyes of the user. The apertures 209 may be controlled by the user 108 and/or the therapist depending on the particular configuration.

The eye movement trackers 210 may be used to track the eye movements of one or more eyes of the user 108. In some examples, the eye movement trackers 210 may take a series of pictures or video of the eyes of the user and, based on various factors (such as the direction of the pupil, iris, and the like), determine the direction to which the eye is pointing and/or moving. In some examples, near-infrared light is directed to the center of the eyes, causing reflections. The reflections are tracked by an infrared camera. In some examples, the eye movement trackers 210 may be used additionally to provide feedback to the user or the therapist when the user does not follow a pointer. The feedback may be audible feedback or may be a message indicating that the eyes are not tracking and/or positive encouragement to follow the pointer. These and other eye movement tracking technologies, such as electro-oculographic technologies using electrodes, may be used and are considered to be within the scope of the presently disclosed subject matter.

The therapy headset 104 further includes display control 212. The display control 212 may include various computing resources, including a power supply, to control a display of the therapy headset 104 as well as provide audio to the therapy audio device 106 in some examples. The display control 212 may act as an interface with the communication device 110 or provide the functionality of the communication device 110. In some examples, the display control 212 may be a location into which a user may insert a cellular telephone or other device to provide a display for the therapy headset 104 in examples in which a display is not integrated into the therapy headset 104.

FIG. 3 illustrates the use of a cellular telephone 300 for use as a display for the therapy headset 104, in accordance with some examples of the presently disclosed subject matter. Illustrated in FIG. 3 is the display enclosure 206. The display enclosure 206 includes slot 302. The slot 302 is an opening of the display enclosure 206 through which a cellular telephone 304 may be inserted. In this manner, a display 306 of the cellular telephone 304 may be used as a display of the therapy headset 104. It should be noted that other manners for inserting or affixing a cellular telephone (or other display device) may be used and are considered to be within the scope of the presently disclosed subject matter. For example, some display enclosures 206 are configured to attach a cellular telephone to an outer section, such as a face 308 of the display enclosure.

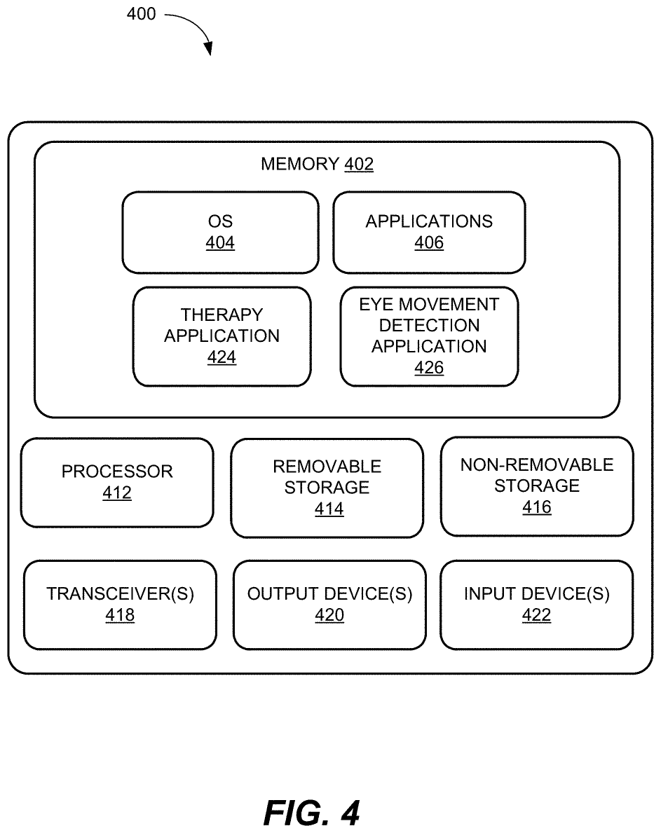

FIG. 4 is an illustration of a therapy computing device 400 for use with the systems and methods described herein, in accordance with some examples of the present disclosure. For example, the therapy computing device 400 may be an onboard computer installed on the therapy headset 104, other components of the therapy device 102, or in communication with various components of the therapy device 102 to provide various functionality described herein. The therapy computing device 400 could be any computing component capable of communicating with or on a cellular network, an internet multimedia subsystem, and/or an IP network. One of skill in the art will recognize that the systems and methods described herein can also be used with a variety of electronic devices, such as, for example, tablet computers, desktops, servers, and other network connected devices.

The therapy computing device 400 can comprise several components to execute various above-mentioned functions. The therapy computing device 400 can comprise memory 402 including an operating system (OS) 804 and one or more standard applications 406. The standard applications 406 can include applications to control the various components of the therapy computing device 400. In this case, the standard applications 406 can also comprise a therapy application 424 and an eye movement detection application 426. The therapy application 424 may be used to instantiate and provide the functionality of the various examples provided herein. For example, when instantiated, the therapy application 424 may cause the initialization of communication with the therapy server 112, may receive instructions from the therapy server 112, and may cause the initialization of the various forms of therapy, such as visual and audio commands. The eye movement detection application 426 may control the operation of the eye movement trackers 210 and may provide eye movement information to the therapy application 424. It should be noted that the therapy application 424 may be configured to provide the functionality of the eye movement detection application 426.

The therapy computing device 400 can also comprise one or more processors 412 and one or more of removable storage 414, non-removable storage 416, transceiver(s) 418, output device(s) 420, and input device(s) 422. In various implementations, the memory 402 can be volatile (such as random access memory (RAM)), non-volatile (such as read only memory (ROM), flash memory, etc.), or some combination of the two. The memory 402 may be used to store various data received from the therapy headset 104, for example.

The memory 402 can also include the OS 404. The OS 404 contains the modules and software that support basic functions, such as scheduling tasks, executing applications, and controlling peripherals. In some examples, the OS 404 can enable the therapy application 424, the eye movement detection application 426, and provide other functions, as described above, via the transceiver(s) 418. The OS 404 can also enable the therapy device 104 to send and retrieve other data and perform other functions. It should be noted that one or more functions of the presently disclosed subject matter may be executed by other systems than the OS 404, such as firmware/FPGA/ASIC.

The therapy computing device 400 can also comprise one or more processors 412. In some implementations, the processor(s) 412 can be a central processing unit (CPU), a graphics processing unit (GPU), both CPU and GPU, or any other processing unit such as an application-specific integrated circuit (ASIC) or Field Programmable Gate Arrays (FPGA), by way of example and not by way of limitation. The therapy computing device 400 may also include additional data storage devices (removable and/or non-removable) such as, for example, magnetic disks, optical disks, or tape. Such additional storage is illustrated in FIG. 4 by removable storage 414 and non-removable storage 416.

Non-transitory computer-readable media may include volatile and nonvolatile, removable and non-removable tangible, physical media implemented in technology for storage of information, such as computer readable instructions, data structures, program modules, or other data. The memory 402, removable storage 414, and non-removable storage 416 are all examples of non-transitory computer-readable media. Non-transitory computer-readable media include, but are not limited to, RAM, ROM, electronically erasable programmable ROM (EEPROM), flash memory or other memory technology, compact disc ROM (CD-ROM), digital versatile discs (DVD) or other optical storage, magnetic cassettes, magnetic tape, magnetic disk storage or other magnetic storage devices, or any other tangible, physical medium which can be used to store the desired information and which can be accessed by the therapy computing device 400. Any such non-transitory computer-readable media may be part of the therapy computing device 400 or may be a separate database, databank, remote server, or cloud-based server.

In some implementations, the transceiver(s) 418 include any transceivers known in the art. In some examples, the transceiver(s) 418 can include wireless modem(s) to facilitate wireless connectivity with other components, the Internet, and/or an intranet, as well as wireless network adapters or other capable equipment.

The transceiver(s) 418 may also include one or more radio transceivers that perform the function of transmitting and receiving radio frequency communications via an antenna (e.g., Wi-Fi or Bluetooth®). In other examples, the transceiver(s) 418 may include wired communication components, such as a wired modem or Ethernet port, for communicating via one or more wired networks.

In some implementations, the output device(s) 420 include any output devices known in the art, such as a display (e.g., a liquid crystal or thin-film transistor (TFT) display), a touchscreen, speakers, a vibrating mechanism, or a tactile feedback mechanism. Thus, the output device(s) can include a screen, or display. The output device(s) 420 can also include speakers, or similar devices, to play sounds or ringtones when an audio call or video call is received. Output device(s) 420 can also include ports for one or more peripheral devices, such as headphones, peripheral speakers, or a peripheral display.

In various implementations, input device(s) 422 include any input devices known in the art. In some examples, the input device(s) 422 may include a camera, a microphone, or a keyboard/keypad or the remote control 120. The input device(s) 422 can include a touch-sensitive display or a keyboard to enable users to enter data and make requests and receive responses via web applications (e.g., in a web browser), make audio and video calls, and use the standard applications 406, among other things. The touch-sensitive display or keyboard/keypad may be a standard push button alphanumeric multi-key keyboard (such as a conventional QWERTY keyboard), virtual controls on a touchscreen, or one or more other types of keys or buttons, and may also include a joystick, wheel, and/or designated navigation buttons, or the like.

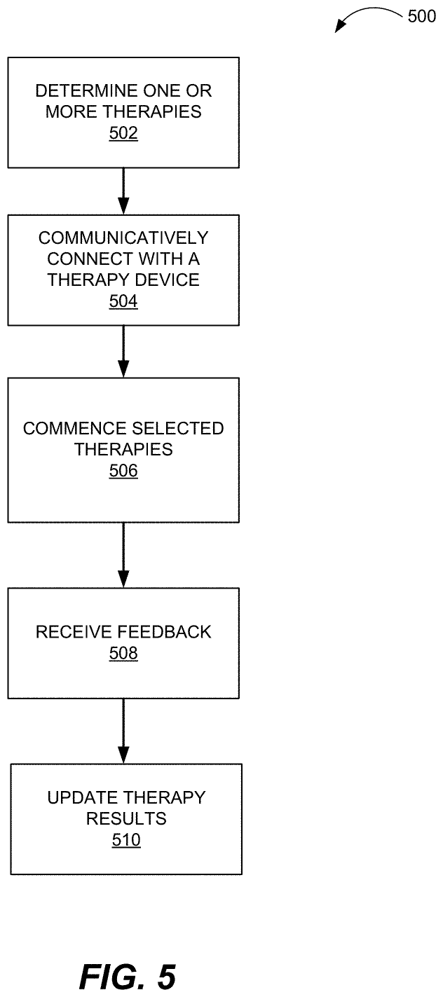

FIG. 5 is a flowchart depicting a process 500 of using the therapy device 102, in accordance with some examples of the present disclosure. The process 500 and other processes described herein are illustrated as example flow graphs, each operation of which may represent a sequence of operations that can be implemented in hardware, software, or a combination thereof. In the context of software, the operations represent computer-executable instructions stored on one or more computer-readable storage media that, when executed by one or more processors, perform the recited operations. Generally, computer-executable instructions include routines, programs, objects, components, data structures, and the like that perform particular functions or implement particular abstract data types. The order in which the operations are described is not intended to be construed as a limitation, and any number of the described operations can be combined in any order and/or in parallel to implement the processes.

Referring to FIG. 5, the process 500 commences operation 502, where one or more therapies for the user 108 are determined. As described above, various therapies may be available for selection from the therapy menu 116. The therapy menu 116 may be updated from input from a therapist or may be updated based on information stored in the therapy results 118 as well as the therapy device 102 itself, such as the capabilities of the therapy device 102.

The process 500 continues to operation 504, where the therapy server 112 is communicatively connected to the therapy device 102. It should be noted that in some examples, the communication connection may be through a network 114. In other examples, the communication connection may be an “offline” connection where the user 108 can download or access therapies selected by a therapist. For example, instead of a therapist doing a therapy in “real time,” there may be situations in which a user may not be able to use an online or active connection. So, in these cases, the user, when able, can download a therapy rather than it be provided in real time. For example, the user can be taking a plane ride. The user can download an “airplane therapy” from the therapist and start that therapy during the plane trip when the user doesn't have access to the Internet.

The presently disclosed subject matter is not limited to an active communication connection, such as a data or voice connection through a cellular network or WIFI network.

The process 500 continues to operation 506, where the selected therapies are commenced. As noted above, various examples of the presently disclosed subject matter provide for multiple therapies to commence at one time, though the presently disclosed subject matter is not so limited, as a single therapy may be commenced.

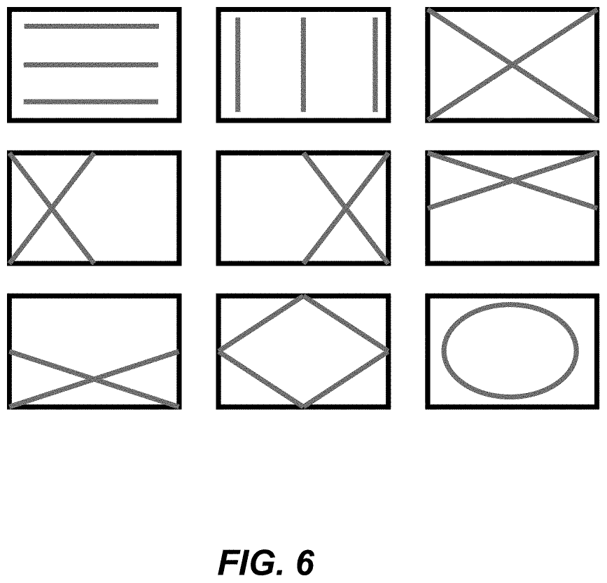

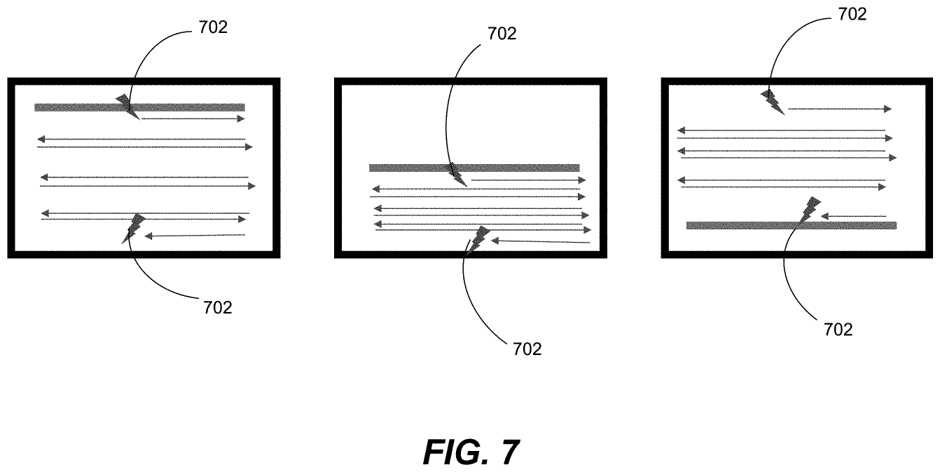

The therapies may vary. For example, one of the therapies may be an EMI session whereby patterns are displayed on a display, patterns such as those illustrated in FIG. 6. The patterns may be visually provided by the movement of a cursor, such as a cursor 702 illustrated in FIG. 7 for the first pattern illustrated in FIG. 6, with the arrows demonstrating a direction of the cursor 702. The patient may be asked to use their eyes and track the patterns as they are displayed or as pointed to by a therapist using a pointer (or a cursor such as the cursor 702 of FIG. 7) on the display, the pointer being movable by the therapist. The patient may be asked about trauma as the patient follows the patterns with their eyes. For each pattern, more than one pass at the pattern may be needed. After each pattern, the patient may be asked if they feel better, worse, or the same. The patterns may be adjusted based on the patient's answers, such as the removal patterns that result in the patient feeling worse. The movement of the patient's eyes are tracked during the process. During this time, various audio or other therapies may be commenced, such as a soft or pleasing sound (e.g. the sound of an ocean). The therapies may also include luminotherapy, whereby the background of the display may be a certain color or picture with the pattern rendered over the color or picture (scene or video). These and other combinations are considered to be within the scope of the presently disclosed subject matter.

Returning to FIG. 5, the process 500 continues to operation 508, where feedback is received. As noted above, the feedback may be audio feedback such as the patient indicating if they feel better, worse, or the same. The feedback may be provided by use of the remote control 120. The feedback may also be biological measurements of the patient from the biologics sensor 122. As noted above, the biologics sensor 122 may be one sensor or may be multiple sensors.

The process 500 continues to operation 510, where the therapy results 118 are updated from information gleaned during the session. The updated therapy results 118 may be used to update the therapy menu 116.

The process 500 thereafter ends.

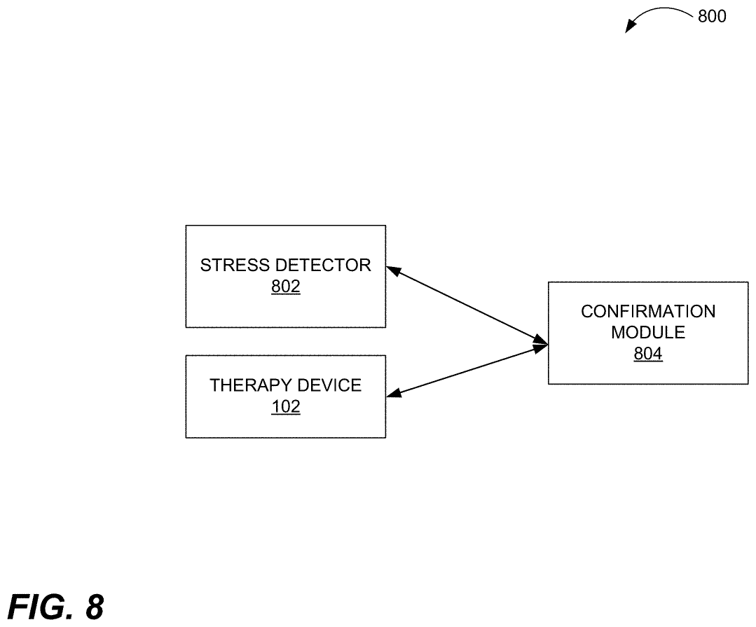

FIG. 8 is an illustration of an example treatment system 800 using a condition detector in conjunction with a therapy device, such as the therapy device 102 of FIG. 1, in accordance with various embodiments of the presently disclosed subject matter. Illustrated in FIG. 8 is a stress detector 802. The stress detector 802 may be a wearable device separate from the therapy device 102 or may be integrated into the therapy device 102. The stress detector 802 is designed to detect one or more biological signals that indicate the onset or the presence of a stressful condition of the person wearing the stress detector 802. The types of biological signals that can be used may vary from patient to patient. For example, heart rate indicators such as electrodes or optical heart rate sensors may be used. In other examples, a respiratory sensor such as Piezo-electric sensor, Acoustic sensor (microphone or laryngophone), Inductive sensor, Capacitive sensor, Hall effect sensor, Radioelectric cartography, Wave cartography, and an Optic based sensor (that measures O2/CO2 levels in the blood or breath) may be utilized. In still further examples, body temperature sensors such as thermocouples, resistance based measurements (platine-based for example), and an infrared sensor may be used.

The stress detector 802, upon the detection of a stress condition of a user wearing or being measured, may communicate with a confirmation module 804. The confirmation module 804 may be a part of the therapy server 112 of FIG. 1 or be a service used by a therapist, or other examples. The communication may be over a network, such as the network 114 of FIG. 1, or other communication technologies. The confirmation module 804 receives the communication from the stress detector 802 and determines if the information in the communication requires a therapeutic process to be commenced. For example, the communication may include biological measurements such as heart rate, body temperature, respiratory, and one or more combinations of these and other biological measurements. In some examples, a therapist could also make a call or teleconsultation with the patient to confirm acute stress

If the confirmation module 804 confirms that a condition exists requiring a therapy, the confirmation module 804 may communicate with the user suggesting that a therapy session take place. Further, the confirmation module 804 may communicate with the therapy device 102 the therapy to be used based on the confirmed condition. For example, a therapist may, at the therapy server 112 or another device, receive the communication from the stress detector 802 and, based on the biological measurements, determine that a combination of relaxation therapy and EMDR. The therapist may cause the transmission of the therapy to the therapy device 102 to allow the patient to commence a therapy in response to the detected stress condition. Further, a teleconsultation between the patient and a therapist may also be included as part of a therapy.

The presently disclosed examples are considered in all respects to be illustrative and not restrictive. The scope of the disclosure is indicated by the appended claims, rather than the foregoing description, and all changes that come within the meaning and range of equivalents thereof are intended to be embraced therein.

What is claimed is:

1. A method of receiving a patient for treatment, the method comprising:
determining at least one therapy;
communicatively connecting to a therapy device;
commencing the at least one therapy; and
receiving feedback from the at least one therapy.

2. The method of claim 1, further comprising updating a therapy result from the feedback received from the at least one therapy.

3. The method of claim 1, wherein the at least one therapy comprises Eye Movement Desensitization and Reprocessing (EMDR), Eye Movement Integration (EMI), relaxation therapy, light therapy, and audio therapy.

4. The method of claim 1, further comprising:
selecting a second therapy; and
initiating the at least one therapy and the second therapy at the same time.

5. The method of claim 1, wherein the therapy device comprises:
a therapy headset comprising:
at least one eye movement sensor for tracking eye movements of a patient; and
a display for displaying images and light to be viewed by the patient; and
a therapy audio device for providing audio for the patient.

6. The method of claim 1, wherein communicatively connecting the therapy device comprises connecting a therapy server to the therapy device over a network.

7. The method of claim 1, wherein receiving feedback from the at least one therapy comprises receiving input from a remote control operated by a patient receiving the at least one therapy.

8. The method of claim 1, wherein receiving feedback from the at least one therapy comprises receiving at least one biological measurement from the patient receiving the at least one therapy.

9. The method of claim 8, wherein the at least one biological measurement comprises electrocardiogram (ECG), Fc, heart rate variability (HRV), respiratory frequency, O2/CO2 expired, arterial pressure, electroretinography, electroencephalogram (EEG), neurofeedback, heart coherence, blood pressure, pulse rate, and body temperature.

10. A non-transitory computer-readable storage medium having computer-executable instructions stored thereupon that, when executed by a computer, cause the computer to perform acts comprising:
determining at least one therapy;
communicatively connecting to a therapy device;
commencing the at least one therapy; and
receiving feedback from the at least one therapy.

11. The non-transitory computer-readable storage medium of claim 10, the computer-executable instructions further causing the computer to perform acts comprising updating a therapy result from the feedback received from the at least one therapy.

12. The non-transitory computer-readable storage medium of claim 10, wherein the at least one therapy comprises Eye Movement Desensitization and Reprocessing (EMDR), Eye Movement Integration (EMI), relaxation therapy, light therapy, and audio therapy.

13. The non-transitory computer-readable storage medium of claim 10, the computer-executable instructions further causing the computer to perform acts comprising:
selecting a second therapy; and
initiating the at least one therapy and the second therapy at the same time.

14. The non-transitory computer-readable storage medium of claim 10, wherein the therapy device comprises:
a therapy headset comprising:
at least one eye movement sensor for tracking eye movements of a patient; and
a display for displaying images and light to be viewed by the patient; and
a therapy audio device for providing audio for the patient.

15. The non-transitory computer-readable storage medium of claim 10, wherein communicatively connecting the therapy device comprises connecting a therapy server to the therapy device over a network.

16. The non-transitory computer-readable storage medium of claim 10, wherein receiving feedback from the at least one therapy comprises receiving input from a remote control operated by a patient receiving the at least one therapy.

17. The non-transitory computer-readable storage medium of claim 10, wherein receiving feedback from the at least one therapy comprises receiving at least one biological measurement from a patient receiving the at least one therapy.

18. The non-transitory computer-readable storage medium of claim 17, wherein the at least one biological measurement comprises electrocardiogram (ECG), Fc, heart rate variability (HRV), respiratory frequency, O2/CO2 expired, arterial pressure, electroretinography, electroencephalogram (EEG), neurofeedback, heart coherence, blood pressure, pulse rate, and body temperature.

19. A system, the system comprising:
a therapy device, comprising:
a therapy headset comprising:
a strap for securing the therapy headset to a patient;
a display for displaying at least one of an image, a pattern, and a color;
a partition for installing at least one aperture to allow the patient to view
the display and at least one eye movement tracker for tracking an eye movement of the patient;
a transceiver for communicatively connecting over a network to a therapy server;
a memory storing computer-executable instructions; and
a processor in communication with the memory, the computer-executable instructions causing the processor to perform acts comprising:
communicatively connecting to the therapy server;
receiving instructions to commence a plurality of therapies;
tracking the eye movement of the patient while the plurality of therapies are being applied;
receiving at least one feedback relating to the plurality of therapies; and
transmitting to the therapy server the at least one feedback; and
a therapy audio device for receiving audio therapy.

20. The system of claim 19, wherein the at least one feedback comprises the eye movement of the patient while the plurality of therapies are being applied, input received from a remote control, an audio feedback provided by the patient, and at least one biological measurement comprising electrocardiogram (ECG), Fc, heart rate variability (HRV), respiratory frequency, O2/CO2 expired, arterial pressure, electroretinography, electroencephalogram (EEG), neurofeedback, heart coherence, blood pressure, pulse rate, and body temperature.

21. The system of claim 19, further comprising a stress detector configured to measure at least one biological signal indicating the presence of a stressful condition.

22. The system of claim 21, wherein the stress detector comprises a heart rate indicator.

23. The system of claim 22, wherein the heart rate indicator comprises an electrode or an optical heart rate sensor.

24. The system of claim 21, wherein the stress detector comprises a respiratory sensor.

25. The system of claim 24, wherein the respiratory sensor comprises a Piezo-electric sensor, an acoustic sensor, an inductive sensor, a capacitive sensor, a Hall effect sensor, Wave cartography, or an optic-based sensor that measures oxygen or carbon dioxide levels in blood or breath.

26. The system of claim 21, wherein the stress detector comprises a body temperature sensor.

27. The system of claim 26, wherein the body temperature sensor comprises a thermocouple, a resistance based measurement, or an infrared sensor.

28. A method of providing a therapy, the method comprising:
determining a first therapy for a patient;
preparing a multi-session treatment to be executed using a therapy device;
communicatively connecting to the therapy device;
causing the therapy device to provide the first therapy for a first treatment session of the multi-session treatment;
receiving feedback from the therapy device on the first treatment session for first therapy; and
modifying a second treatment session of the multi-session treatment until a condition of the patient is reached.

29. The method of claim 28, wherein the therapy device comprises:
a therapy headset comprising:
at least one eye movement sensor for tracking eye movements of a patient; and
a display for displaying images and light to be viewed by the patient; and
a therapy audio device for providing audio for the patient.

30. A method of providing a therapy, the method comprising:
receiving a communication from a stress detector;
confirming a condition exists based on the communication that requires the therapy;
selecting the therapy based on the condition;
transmitting the therapy to be applied to a therapy device.

31. The method of claim 30, wherein the therapy device comprises:
a therapy headset comprising:
at least one eye movement sensor for tracking eye movements of a patient; and
a display for displaying images and light to be viewed by the patient; and
a therapy audio device for providing audio for the patient.

32. The method of claim 30, wherein the condition is a heart rate, wherein the stress detector comprises a heart rate indicator.

33. The method of claim 32, wherein the heart rate indicator comprises an electrode or an optical heart rate sensor.

34. The method of claim 30, wherein the condition comprises a respiratory condition, wherein the stress detector comprises a respiratory sensor.

35. The method of claim 34, wherein the respiratory sensor comprises a Piezo-electric sensor, an acoustic sensor, an inductive sensor, a capacitive sensor, a Hall effect sensor, Wave cartograph, or an optic-based sensor that measures oxygen or carbon dioxide levels in blood or breath.

36. The method of claim 30, wherein the condition comprises a body temperature, wherein the stress detector comprises a body temperature sensor.

37. The method of claim 36, wherein the body temperature sensor comprises a thermocouple, a resistance based measurement, or an infrared sensor.

38. The method of claim 30, wherein confirming a condition exists based on the communication that requires the therapy comprises a call between a therapist and a patient receiving the therapy.
